(19) United States
(12) Patent Application Publication
(10) Pub. No.: US 2018/0133203 A1
(43) Pub. Date: May 17, 2018

(54) METHODS OF TREATING LIVER DISEASE

(71) Applicant: Gilead Sciences, Inc., Foster City, CA (US)

(72) Inventors: David Gordon Clarkson Breckenridge, San Mateo, CA (US); Grant Raymond Budas, Palo Alto, CA (US); John T. Liles, San Jose, CA (US); William J. Watkins, Saratoga, CA (US)

(21) Appl. No.: 15/796,195

(22) Filed: Oct. 27, 2017

Related U.S. Application Data
(60) Provisional application No. 62/482,102, filed on Apr. 5, 2017.
(60) Provisional application No. 62/481,445, filed on Apr. 4, 2017.
(60) Provisional application No. 62/421,090, filed on Nov. 11, 2016.

(51) Int. Cl. A61K 31/4439; A61P 1/16

(52) U.S. Cl. CPC A61K 31/4439; A61K 2300/00; A61P 1/16

(57) ABSTRACT

The present disclosure relates to a method of preventing and/or treating liver disease comprising administering an ASK1 inhibitor in combination with a FXR agonist to a patient in need thereof.

### CROSS-REFERENCE TO RELATED APPLICATIONS

This application claims the benefit of U.S. Provisional Application Ser. No. 62/421,090, filed Nov. 11, 2016; U.S. Provisional Application Ser. No. 62/481,445, filed Apr. 4, 2017; and, U.S. Provisional Application Ser. No. 62/482,102, filed Apr. 5, 2017. The entireties of these applications are incorporated herein by reference.

### FIELD

The present disclosure relates to methods of preventing and/or treating liver diseases.

### BACKGROUND

Liver disease is generally classified as acute or chronic based upon the duration of the disease. Liver disease may be caused by infection, injury, exposure to drugs or toxic compounds, alcohol, impurities in foods, and the abnormal build-up of normal substances in the blood, an autoimmune process, a genetic defect (such as haemochromatosis), or unknown cause(s).

Liver disease is a leading cause of death worldwide. In particular, it has been seen that a diet high in fat damages the liver in ways that are surprisingly similar to hepatitis. The American Liver Foundation estimates that more than 20 percent of the population has non-alcoholic fatty liver disease (NAFLD). It is suggested that obesity, unhealthy diets, and sedentary lifestyles may contribute to the high prevalence of NAFLD. When left untreated, NAFLD can progress to non-alcoholic steatohepatitis (NASH) causing serious adverse effects. Once NASH is developed, it would cause the liver to swell and scar (i.e. cirrhosis) over time.

Although preliminary reports suggest positive lifestyle changes could prevent or reverse liver damage, there are no effective medical treatments for NAFLD. Accordingly, there remains a need to provide new effective pharmaceutical agents to treat liver diseases.

### SUMMARY

Disclosed herein is a method of treating and/or preventing liver disease in a patient in need thereof, comprising administering to the patient a therapeutically effective amount of an apoptosis signal regulating kinase 1 (ASK1) inhibitor in combination with a therapeutically effective amount of farnesoid X receptor (FXR) agonist. The liver disease can be any liver disease, including, but not limited to, chronic and/or metabolic liver diseases, nonalcoholic fatty liver disease (NAFLD), and nonalcoholic steatohepatitis (NASH).

In certain embodiments, provided herein is a method of treating and/or preventing nonalcoholic steatohepatitis (NASH) in a patient in need thereof, comprising administering to the patient a therapeutically effective amount of an ASK1 inhibitor in combination with a therapeutically effective amount of a FXR agonist.

In the methods provided herein, the ASK1 inhibitor and the FXR agonist can be coadministered. In such embodiments, the ASK1 inhibitor and the FXR agonist can be administered together as a single pharmaceutical composition, or separately in more than one pharmaceutical composition. Accordingly, also provided herein is a pharmaceutical composition comprising a therapeutically effective amount of an ASK1 inhibitor and a therapeutically effective amount of a FXR agonist.

### DESCRIPTION OF THE FIGURES

FIG. 1: PSR staining (% area of liver) quantified by morphometric image analysis. Graph shows mean±SEM.

FIG. 2: Hydroxyproline content of the liver expressed as micrograms of hydroxyproline per grams of liver tissue. Graph shows mean±SEM.

FIG. 3: Desmin⁺ staining (% area of liver) quantified by morphometric image analysis. Graph shows mean±SEM.

FIG. 4: Hepatic expression of liver fibrosis genes Col1a1 measured by quantitative RT-PCR. Graph shows mean±SEM.

FIG. 5: Hepatic expression of liver fibrosis genes TIMP-1 measured by quantitative RT-PCR. Graph shows mean±SEM.

FIG. 6: Hepatic steatosis (% vacuolation area of liver) quantified by morphometric image analysis. Graph shows mean±SEM.

FIG. 7: Hepatic cholesterol content of the liver expressed as milligrams of cholesterol per grams of liver tissue. Graph shows mean±SEM.

FIG. 8: Total level of Bile Acids in the plasma expressed as nanograms of bile acids per milliliter of plasma. Graph shows mean±SEM.

FIG. 9: Hepatic expression of the inflammation gene IL1-β in the liver measured by quantitative nanostring. Graph shows mean±SEM.

### DETAILED DESCRIPTION

### Definitions and General Parameters

As used in the present specification, the following terms and phrases are generally intended to have the meanings as set forth below, except to the extent that the context in which they are used indicates otherwise.

As used herein, the term “about” used in the context of quantitative measurements means the indicated amount±10%, or alternatively the indicated amount±5% or ±1%.

The term “pharmaceutically acceptable salt” refers to a salt of a compound disclosed herein that retains the biological effectiveness and properties of the underlying compound, and which is not biologically or otherwise undesirable. There are acid addition salts and base addition salts. Pharmaceutically acceptable acid addition salts may be prepared from inorganic and organic acids.

Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, at al. Journal of Pharmaceutical Science, January 1977 vol. 66, No. 1, and other sources.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The terms “therapeutically effective amount” and “effective amount” are used interchangibly and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt of formula (I) for the purpose of: (i) delaying the onset of a disease, that is, causing the clinical symptoms of the disease not to develop or delaying the development thereof; (ii) inhibiting the disease, that is, arresting the development of clinical symptoms; and/or (iii) relieving the disease, that is, causing the regression of clinical symptoms or the severity thereof.

### Liver Diseases

Liver diseases are acute or chronic damages to the liver based in the duration of the disease. The liver damage may be caused by infection, injury, exposure to drugs or toxic compounds such as alcohol or impurities in foods, an abnormal build-up of normal substances in the blood, an autoimmune process, a genetic defect (such as haemochromatosis), or other unknown causes. Exemplary liver diseases include, but are not limited to, cirrhosis, liver fibrosis, non-alcoholic fatty liver disease (NAFLD), non-alcoholic steatohepatitis (NASH), alcoholic steatohepatitis (ASH), hepatic ischemia reperfusion injury, primary biliary cirrhosis (PBC), and hepatitis, including both viral and alcoholic hepatitis.

Non-alcoholic fatty liver disease (NAFLD) is the build up of extra fat in liver cells that is not caused by alcohol. NAFLD may cause the liver to swell (i.e. steatohepatitis), which in turn may cause scarring (i.e. cirrhosis) over time and may lead to liver cancer or liver failure. NAFLD is characterized by the accumulation of fat in hepatocyes and is often associated with some aspects of metabolic syndrome (e.g. type 2 diabetes mellitus, insulin resistance, hyperlipidemia, hypertension). The frequency of this disease has become increasingly common due to consumption of carbohydrate-rich and high fat diets. A subset (˜20%) of NAFLD patients develop nonalcoholic steatohepatitis (NASH).

NASH, a subtype of fatty liver disease, is the more severe form of NAFLD. It is characterized by macrovesicular steatosis, balloon degeneration of hepatocytes, and/or inflammation ultimately leading to hepatic scarring (i.e. fibrosis). Patients diagnosed with NASH progress to advanced stage liver fibrosis and eventually cirrhosis. The current treatment for cirrhotic NASH patients with end-stage disease is liver transplant.

A study has shown that a significant proportion of diagnosed NASH patients (39%) have not had a liver biopsy to confirm the diagnosis. A greater proportion of diagnosed NASH patients have metabolic syndrome parameters than what is reported in the literature (type-II diabetes mellitus 54%, Obesity 71%, metabolic syndrome 59%). 82% of physicians use a lower threshold value to define significant alcohol consumption compared with practice guideline recommendations. 88% of physicians prescribe some form of pharmacologic treatment for NASH (Vit E: prescribed to 53% of NASH patients, statins: 57%, metformin: 50%). Therefore, the vast majority of patients are prescribed medications despite a lack of a confirmed diagnosis or significant data to support the intervention and alcohol thresholds to exclude NASH are lower than expected.

Another common liver disease is primary sclerosing cholangitis (PSC). It is a chronic or long-term liver disease that slowly damages the bile ducts inside and outside the liver. In patients with PSC, bile accumulates in the liver due to blocked bile ducts, where it gradually damages liver cells and causes cirrhosis, or scarring of the liver. Currently, there is no effective treatment to cure PSC. Many patients having PSC ultimately need a liver transplant due to liver failure, typically about 10 years after being diagnosed with the disease. PSC may also lead to bile duct cancer.

Liver fibrosis is the excessive accumulation of extracellular matrix proteins, including collagen, that occurs in most types of chronic liver diseases. Advanced liver fibrosis results in cirrhosis, liver failure, and portal hypertension and often requires liver transplantation.

### Methods

Disclosed herein is a method of treating and/or preventing liver disease in a patient in need thereof, comprising administering to the patient a therapeutically effective amount of an ASK1 inhibitor in combination with a therapeutically effective amount of a FXR agonist. The presence of active liver disease can be detected by the existence of elevated enzyme levels in the blood. Specifically, blood levels of alanine aminotransferase (ALT) and aspartate aminotransferase (AST) above clinically accepted normal ranges are known to be indicative of on-going liver damage. Routine monitoring of liver disease patients for blood levels of ALT and AST is used clinically to measure progress of the liver disease while on medical treatment. Reduction of elevated ALT and AST to within the accepted normal range is taken as clinical evidence reflecting a reduction in the severity of the patient's on-going liver damage.

In certain embodiments, the liver disease is a chronic liver disease. Chronic liver diseases involve the progressive destruction and regeneration of the liver parenchyma, leading to fibrosis and cirrhosis. In general, chronic liver diseases can be caused by viruses (such as hepatitis B, hepatitis C, cytomegalovirus (CMV), or Epstein Barr Virus (EBV)), toxic agents or drugs (such as alcohol, methotrexate, or nitrofurantoin), a metabolic disease (such as non-alcoholic fatty liver disease (NAFLD), non-alcoholic steatohepatitis (NASH), haemochromatosis, or Wilson's Disease), an autoimmune disease (such as Autoimmune Chronic Hepatitis, Primary Biliary Cholangitis (formerly known as Primary Biliary Cirrhosis), or Primary Sclerosing Cholangitis), or other causes (such as right heart failure).

In one embodiment, provided herein is a method for reducing the level of cirrhosis. In one embodiment, cirrhosis is characterized pathologically by loss of the normal microscopic lobular architecture, with fibrosis and nodular regeneration. Methods for measuring the extent of cirrhosis are well known in the art. In one embodiment, the level of cirrhosis is reduced by about 5% to about 100%. In one embodiment, the level of cirrhosis is reduced by at least about 5%, at least about 10%, at least about 15%, at least about 20%, at least about 25%, at least about 30%, at least about 35%, at least about 40%, at least about 45%, at least 50%, at least about 55%, at least about 60%, at least about 65%, at least about 70%, at least about 75%, at least about 80%, at least about 85%, at least about 90%, at least about 95%, or about 100% in the subject.

In certain embodiments, the liver disease is a metabolic liver disease. In one embodiment, the liver disease is non-alcoholic fatty liver disease (NAFLD). NAFLD is associated with insulin resistance and metabolic syndrome (obesity, combined hyperlipidemia, diabetes mellitus (type II) and high blood pressure). NAFLD is considered to cover a spectrum of disease activity, and begins as fatty accumulation in the liver (hepatic steatosis).

It has been shown that both obesity and insulin resistance probably play a strong role in the disease process of NAFLD. In addition to a poor diet, NAFLD has several other known causes. For example, NAFLD can be caused by certain medications, such as amiodarone, antiviral drugs (e.g., nucleoside analogues), aspirin (rarely as part of Reye's syndrome in children), corticosteroids, methotrexate, tamoxifen, or tetracycline. NAFLD has also been linked to the consumption of soft drinks through the presence of high fructose corn syrup which may cause increased deposition of fat in the abdomen, although the consumption of sucrose shows a similar effect (likely due to its breakdown into fructose). Genetics has also been known to play a role, as two genetic mutations for this susceptibility have been identified.

If left untreated, NAFLD can develop into non-alcoholic steatohepatitis (NASH), which is the most extreme form of NAFLD, a state in which steatosis is combined with inflammation and fibrosis. NASH is regarded as a major cause of cirrhosis of the liver. Accordingly, provided herein is a method of treating and/or preventing nonalcoholic steatohepatitis (NASH) in a patient in need thereof, comprising administering to the patient a therapeutically effective amount of an ASK1 inhibitor in combination with a therapeutically effective amount of a a FXR agonist.

Also provided herein is a method of treating and/or preventing liver fibrosis in a patient in need thereof, comprising administering to the patient a therapeutically effective amount of an ASK1 inhibitor in combination with a therapeutically effective amount of a FXR agonist. Liver fibrosis is the excessive accumulation of extracellular matrix proteins including collagen that occurs in most types of chronic liver diseases. In certain embodiments, advanced liver fibrosis results in cirrhosis and liver failure. Methods for measuring liver histologies, such as changes in the extent of fibrosis, lobular hepatitis, and periportal bridging necrosis, are well known in the art.

In one embodiment, the level of liver fibrosis, which is the formation of fibrous tissue, fibroid or fibrous degeneration, is reduced by more that about 90%. In one embodiment, the level of fibrosis, which is the formation of fibrous tissue, fibroid or fibrous degeneration, is reduced by at least about 90%, at least about 80%, at least about 70%, at least about 60%, at least about 50%, at least about 40%, at least about 30%, at least about 20%, at least about 10%, at least about 5% or at least about 2%.

In one embodiment, the compounds provided herein reduce the level of fibrogenesis in the liver. Liver fibrogenesis is the process leading to the deposition of an excess of extracellular matrix components in the liver known as fibrosis. It is observed in a number of conditions such as chronic viral hepatitis B and C, alcoholic liver disease, drug-induced liver disease, hemochromatosis, autoimmune hepatitis, Wilson disease, Primary Biliary Cholangitis (formerly known as Primary Biliary Cirrhosis), sclerosing cholangitis, liver schistosomiasis and others. In one embodiment, the level of fibrogenesis is reduced by more that about 90%. In one embodiment, the level of fibrogenesis is reduced by at least about 90%, at least about 80%, at least about 70%, at least about 60%, at least about 50%, at least 40%, at least about 30%, at least about 20%, at least about 10%, at least about 5% or at least 2%.

In still other embodiments, provided herein is a method of treating and/or preventing primary sclerosing cholangitis (PSC) in a patient in need thereof, comprising administering to the patient a therapeutically effective amount of an ASK1 inhibitor in combination with a therapeutically effective amount of a FXR agonist.

It has been observed that patients having NASH are on average about 2.8 years older than healthy patients in epigenetic testing. Thus, in one embodiment, compounds useful for the treatment of NASH would be useful for slowing, improving or reversing epigenetic age or effects of aging due to NASH. In another embodiment, combination therapies for the treatment of NASH such as, for example, the combination of an ASK1 inhibitor with an FXR agonist as disclosed herein may be useful for improvement or reversal of aging effects due to NASH.

In one embodiment, the ASK1 inhibitor and the FXR agonist may be administered together in a combination formulation or in separate pharmaceutical compositions, where each inhibitor may be formulated in any suitable dosage form. In certain embodiments, the methods provided herein comprise administering separately a pharmaceutical composition comprising an ASK1 inhibitor and a pharmaceutically acceptable carrier or excipient and a pharmaceutical composition comprising a FXR agonist and a pharmaceutically acceptable carrier or excipient. Combination formulations according to the present disclosure comprise an ASK1 inhibitor and a FXR agonist together with one or more pharmaceutically acceptable carriers or excipients and optionally other therapeutic agents. Combination formulations containing the active ingredient may be in any form suitable for the intended method of administration.

### ASK1 Inhibitors

In certain embodiments of the methods and pharmaceutical compositions disclosed herein, the ASK1 inhibitor is a compound having the structure of Formula (I):

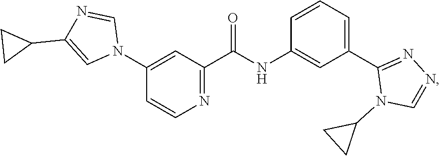 [SMILES: O=C(Nc1cccc(-c2nncn2C2CC2)c1)c1cc(-n2cnc(C3CC3)c2)ccn1]

or a pharmaceutically acceptable salt thereof.

In certain embodiments of the methods and pharmaceutical compositions disclosed herein, the ASK1 inhibitor is a compound having the structure of Formula (II):

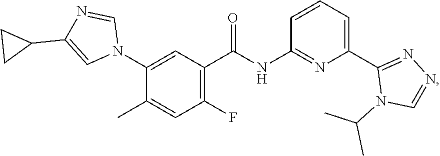 [SMILES: Cc1cc(F)c(C(=O)Nc2cccc(-c3nncn3C(C)C)n2)cc1-n1cnc(C2CC2)c1]

or a pharmaceutically acceptable salt thereof.

The compounds of Formula (I) and Formula (II) may be synthesized and characterized using methods known to those of skill in the art, such as those described in U.S. Patent Application Publication Nos. 2011/0009410 and 2013/0197037. In one embodiment, the ASK1 inhibitor is the compound of Formula (I) or a pharmaceutically acceptable salt thereof. In one embodiment, the ASK1 inhibitor is the compound of Formula (II) or a pharmaceutically acceptable salt thereof.

### FXR Agonist

In certain embodiments of the methods and pharmaceutical compositions disclosed herein, the FXR agonist is a compound having the structure of Formula (III):

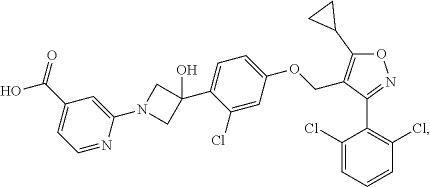 [SMILES: O=C(O)c1ccnc(N2CC(O)(c3ccc(OCc4c(-c5c(Cl)cccc5Cl)noc4C4CC4)cc3Cl)C2)c1]

or a pharmaceutically acceptable salt thereof.

In certain embodiments of the methods and pharmaceutical compositions disclosed herein, the FXR agonist is a compound having the structure of Formula (IV):

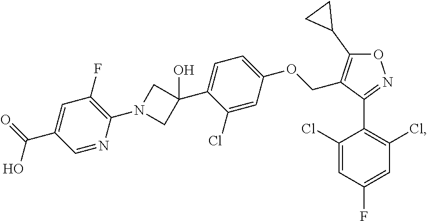 [SMILES: O=C(O)c1cnc(N2CC(O)(c3ccc(OCc4c(-c5c(Cl)cc(F)cc5Cl)noc4C4CC4)cc3Cl)C2)c(F)c1]

or a pharmaceutically acceptable salt thereof.

The compounds of Formula (III) and Formula (IV) may be synthesized and characterized using methods known to those of skill in the art, such as those described in U.S. Publication No. 2014/0221659.

### Dosing and Administration

While it is possible for an active ingredient to be administered alone, it may be preferable to present them as pharmaceutical formulations or pharmaceutical compositions as described below. The formulations, both for veterinary and for human use, of the disclosure comprise at least one of the active ingredients, together with one or more acceptable carriers therefor and optionally other therapeutic ingredients. The carrier(s) must be “acceptable” in the sense of being compatible with the other ingredients of the formulation and physiologically innocuous to the recipient thereof.

Each of the active ingredients can be formulated with conventional carriers and excipients, which will be selected in accord with ordinary practice. Tablets can contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

The therapeutically effective amount of active ingredient can be readily determined by a skilled clinician using conventional dose escalation studies. Typically, the active ingredient will be administered in a dose from 0.01 milligrams to 2 grams. In one embodiment, the dosage will be from about 10 milligrams to 450 milligrams. In another embodiment, the dosage will be from about 25 to about 250 milligrams. In another embodiment, the dosage will be about 50 or 100 milligrams. In one embodiment, the dosage will be about 100 milligrams. In one embodiment, 18 mg of an ASK1 inhibitor is administered. In a specific embodiment, 18 mg of the compound of Formula (II) is administered. In one embodiment, 30 mg of an FXR agonist is administered. In a specific embodiment, 30 mg of the compound of Formula (III) is administered. It is contemplated that the active ingredient may be administered once, twice or three times a day. Also, the active ingredient may be administered once or twice a week, once every two weeks, once every three weeks, once every four weeks, once every five weeks, or once every six weeks.

The pharmaceutical composition for the active ingredient can include those suitable for the foregoing administration routes. The formulations can conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations suitable for oral administration can be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste. In certain embodiments, the active ingredient may be administered as a subcutaneous injection.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, preservative, or surface active agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

The active ingredient can be administered by any route appropriate to the condition. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. In certain embodiments, the active ingredients are orally bioavailable and can therefore be dosed orally. In one embodiment, the patient is human.

When used in combination in the methods disclosed herein, the ASK1 inhibitor and the FXR agonist can be administered together in a single pharmaceutical composition or seperately (either concurrently or sequentially) in more than one pharmaceutical composition. In certain embodiments, the ASK1 inhibitor and the FXR agonist are administered together. In other embodiments, the ASK1 inhibitor and the FXR agonist are administered separately. In some aspects, the ASK1 inhibitor is administered prior to the FXR agonist. In some aspects, the FXR agonist is administered prior to the ASK1 inhibitor. When administered separately, the ASK1 inhibitor and the FXR agonist can be administered to the patient by the same or different routes of delivery.

### Pharmaceutical Compositions

The pharmaceutical compositions of the disclosure comprise an effective amount of an ASK1 inhibitor selected from the group consisting of a compound of Formula (I) and a compound of Formula (II), and an effective amount of a FXR agonist selected from the group consisting of a compound of Formula (III) and a compound of Formula (IV).

When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as, for example, calcium or sodium carbonate, lactose, lactose monohydrate, croscarmellose sodium, povidone, calcium or sodium phosphate; granulating and disintegrating agents, such as, for example, maize starch, or alginic acid; binding agents, such as, for example, cellulose, microcrystalline cellulose, starch, gelatin or acacia; and lubricating agents, such as, for example, magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as, for example, glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as, for example, peanut oil, liquid paraffin or olive oil.

Aqueous suspensions of the disclosure contain the active materials in admixture with excipients suitable for the manufacture of aqueous suspensions. Such excipients include a suspending agent, such as, for example, sodium carboxymethylcellulose, methylcellulose, hydroxypropyl methylcelluose, sodium alginate, polyvinylpyrrolidone, gum tragacanth and gum acacia, and dispersing or wetting agents such as, for example, a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as, for example, ethyl or n-propyl p-hydroxy-benzoate, one or more coloring agents, one or more flavoring agents and one or more sweetening agents, such as, for example, sucrose or saccharin.

Oil suspensions may be formulated by suspending the active ingredient in a vegetable oil, such as, for example, arachis oil, olive oil, sesame oil or coconut oil, or in a mineral oil such as, for example, liquid paraffin. The oral suspensions may contain a thickening agent, such as, for example, beeswax, hard paraffin or cetyl alcohol. Sweetening agents, such as, for example, those set forth above, and flavoring agents may be added to provide a palatable oral preparation. These compositions may be preserved by the addition of an antioxidant such as, for example, ascorbic acid.

Dispersible powders and granules of the disclosure suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the disclosure may also be in the form of oil-in-water emulsions. The oily phase may be a vegetable oil, such as, for example, olive oil or arachis oil, a mineral oil, such as, for example, liquid paraffin, or a mixture of these. Suitable emulsifying agents include naturally-occurring gums, such as, for example, gum acacia and gum tragacanth, naturally occurring phosphatides, such as, for example, soybean lecithin, esters or partial esters derived from fatty acids and hexitol anhydrides, such as, for example, sorbitan monooleate, and condensation products of these partial esters with ethylene oxide, such as, for example, polyoxyethylene sorbitan monooleate. The emulsion may also contain sweetening and flavoring agents. Syrups and elixirs may be formulated with sweetening agents, such as, for example, glycerol, sorbitol or sucrose. Such formulations may also contain a demulcent, a preservative, a flavoring or a coloring agent.

The pharmaceutical compositions of the disclosure may be in the form of a sterile injectable preparation, such as, for example, a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as, for example, a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as, for example, oleic acid may likewise be used in the preparation of injectables.

The amount of active ingredient that may be combined with the carrier material to produce a single dosage form will vary depending upon the host treated and the particular mode of administration, such as oral administration or subcutaneous injection. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur. When formulated for subcutaneous administration, the formulation is typically administered about twice a month over a period of from about two to about four months.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations can be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

### Examples

### Example 1. Efficacy in a Rat Model of NASH

The following study was conducted to evaluate the efficacy of the combination of an ASK1 inhibitor and an FXR agonist in a rodent model of non-alcoholic steatohepatitis (NASH), relative to the efficacy of the individual agents alone in the model. NASH was induced in male Wistar rats by administration of a choline-deficient high fat diet (CDHFD) combined with the chronic administration of sodium nitrite (CDHFD/NaNO₂). The model capitalizes on the “two hit theory” of NASH by inducing both metabolic dysfunction (CDHFD) and oxidative stress (NaNO₂) in the liver to result in hepatic fibrosis that is similar in character and severity to that seen in patients with advanced NASH.

Rats were fed CDHFD for a total of 14 weeks and were administered NaNO₂ from week 4 to 14. The compound of Formula (III) (given as admixture in diet, adjusted to deliver 30 mg/kg/day), compound of Formula (I) (administered as a 0.2% admixture in diet), or vehicle was administered from week 4 to 14. The following endpoints were assessed at the completion of the 14 week study: i) liver fibrosis measured by liver hydroxyproline (OHP) content and by quantitative morphometric analysis of collagen content by Picosirius red (PSR) staining; ii) myofibroblast activation in liver sections as measured by quantitative IHC of the myofibroblast marker desmin; and, iii) Col1a1 and Timp1 in liver tissue measured by RT-PCR.

### Methods

### Animals

Male, Wistar rats (aged 6-8 weeks at study inception) and used in the study. All animals were housed under standard vivarium conditions and allowed to acclimate for 14 days prior to study initiation.

### In-Life Experimental Protocol for the CDHFD/NaNO₂ Rat Model

The experimental design is shown in Table 1. All CDHFD/NaNO₂ animals were fed a choline-deficient high-fat diet (CDHFD; Research Diets, Inc) for 14 weeks and were also administered intraperitoneal injections of NaNO₂ (3 injections per week; Sigma Aldrich #31443) at a dose of 25 mg/kg (from week 4 to 10) and at a dose of 12.5 mg/kg (from week 10 to 14).

Formula (III) (given as admixture in the CDHFD diet, adjusted to deliver 30 mg/kg/day, n=10 animals per group) and Formula (I) (given as a 0.2% admixture in the CDHFD diet, n=10 per group) were administered drug from week 4 to 14. Vehicle control animals were administered the same CDHFD diet with no drug added (Vehicle; n=10 animals per group) from week 4 to 14. Healthy control animals were fed a normal chow diet and did not receive NaNO₂ (control, n=10 per group). On the final day of study, rats received a single oral gavage of either Formula (I) or Formula (III) at a dose of 30 mg/kg, four hours before animals were euthanized and tissues were collected for analyses. All endpoints were assessed at the completion of the 14 week study protocol.

TABLE 1
Experimental Design and Dose Groups

| | | | | | | Final Oral Dose |
|---|---|---|---|---|---|---|
| | | Diet | NaNO₂ | | Diet Admix Dose | (4 hours before |
| Group | n | (week 1-14) | (wk 4-14) | Treatment | (week 4-14) | tissue collection) |
| 1 | 10 | Normal Chow | No | N/A | N/A | N/A |
| 2 | 10 | CDHFD | Yes | Vehicle | N/A | N/A |
| 3 | 10 | CDHFD | Yes | Formula (I) | 0.2% in chow | 30 mg/kg |
| 4 | 10 | CDHFD | Yes | Formula (III) | 30 mg/kg/day | 30 mg/kg |
| 5 | 10 | CDHFD | Yes | Formula (I) | 0.2% in chow | 30 mg/kg |
| | | | | Formula (III) | 30 mg/kg/day | 30 mg/kg |

### Quantitative Morphometric Analysis of PSR and Desmin IHC

Whole slide images of Picrosirius Red (PSR) and Desmin stained slides were captured using a Leica AT2 scanner at 40× magnification. Digital slide images were checked for scanning quality, annotated and exported to appropriate network folders within Leica Digital Image Hub archive. Quantitative image analysis was performed on the whole slide-scan images using Definiens Tissue Studio Architect XD (Definiens Inc.) to determine the extent and intensity of PSR and Desmin. The Definiens Composer function was used to distinguish liver tissue from the surrounding glass slide and to isolate and remove optical anomalies and tissue defects. The total PSR-stained area and Desmin IHC staining were measured and expressed as a percentage of total liver area stained.

Gene Expression by qRT-PCR

Two target organs, the ileum and the liver, were subjected to gene expression analysis by qRT-PCR. Total RNA from 25 mg ground frozen tissue was isolated using RNAzol RT Reagent (Sigma Aldrich, Cat No #R4533) and a RNA Isolation Kit (Qiagen, Cat No #74182) following the manufacturer's instructions. cDNAs were synthesized from 0.5 μg of total RNA using SuperscriptII™ reverse transcriptase (Life Technologies, Cat No #18064-014) primed with 50 pmol of random hexamers. Quantitative PCR was performed and analyzed using Absolute QPCR Rox Mix (Life Technologies, Cat No # AB-1132) and a 384-format ABI 7900HT Sequence Detection System (Applied Biosystems). In liver tissue reverse and forward specific primers and probe (Integraded DNA Technologies, USA) were used for expression analysis of Col1A1 and TIMP1.

### Liver Hydroxyproline Count

Liver hydroxyproline was quantified by an enzymatic method by the reaction of oxidized hydroxyproline with 4-(dimethylamino)benzaldehyde (DMAB), which results in a colorimetric (560 nm) product proportional to the hydroxyproline present. Snap frozen liver samples were powdered under liquid nitrogen then homogenized in water (100 μl/10 mg) and hydrolyzed for 18 h at 120° C. in 12 N HCl solution. After hydrolysis was completed, samples were centrifuged for 5 min at 13,000 g at room temperature then transferred to a 96 well plate and dried at 60° C. Dried samples were oxidized with 100 μl chloramine-T, and incubated for 20 minutes at room temperature. 100 μl of freshly-made DMAB solution was added to each well and the samples were incubated for 30 minutes. The hydroxyproline content was determined photometrically by measuring the absorbance at 560 nm. A standard curve was used to determine hydroxyproline content for each sample.

### Results

### Liver OHP Content and Quantitative Morphometric Analysis of Collagen by PSR

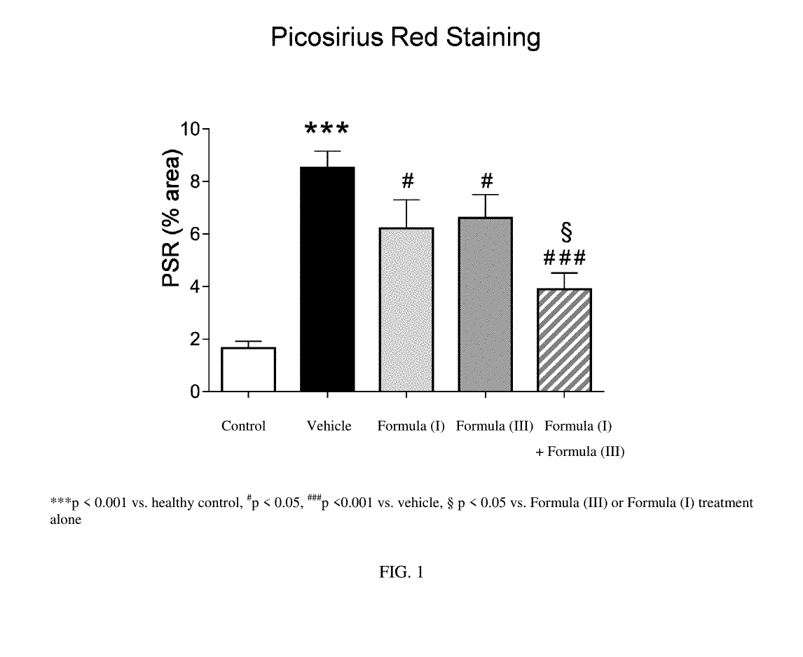

Histological liver sections were stained for Picrosirius Red (PSR) to visualize liver collagen content following 12 weeks of CDHFD/NaNO₂. PSR staining was quantified by morphometric image analysis. The data are shown in FIG. 1. Rats administered CDHFD/NaNO₂ had a 5-fold increase in liver collagen when compared to healthy control rats (PSR area increased from 1.7±0.3% in healthy control rats to 8.5±0.6% in vehicle-treated CDHFD/NaNO₂ rats, p<0.001). Treatment with the compound of Formula (I) reduced liver collagen by 27% (PSR area was reduced from 8.5±0.6% in vehicle-treated CDHFD/NaNO₂ rats to 6.2±1.1%, p<0.05). Treatment with the compound of Formula (III) reduced liver collagen by 22% (PSR area was reduced from 8.5±0.6% in vehicle-treated CDHFD/NaNO2 rats to 6.6±0.9%, p<0.05). Treatment with the combination of the compound of Formula (I) and the compound of Formula (III) reduced collagen by 54% (PSR area was reduced from 8.5±0.6% in vehicle-treated CDHFD/NaNO2 rats to 3.9±0.6%, p<0.001). The effect of combination treatment of the compound of Formula (I) and the compound of Formula (III) to reduce liver collagen was significantly better than either agent administered alone (p<0.05).

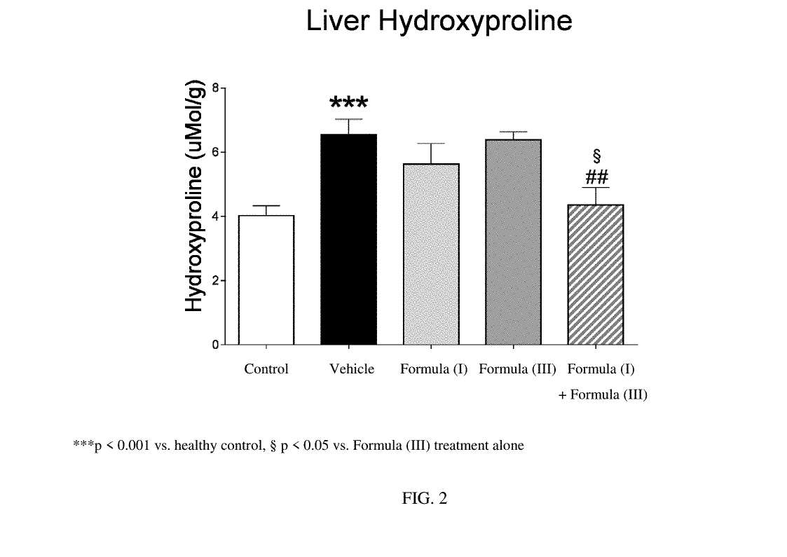

Biochemical assessment of liver fibrosis was performed by measuring liver hydroxyproline content. The data are shown in FIG. 2. Rats administered CDHFD/NaNO₂ had a 1.6-fold increase in liver hydroxyproline content when compared to healthy control rats (liver hydroxyproline increased from 4.0±0.3 μmol/g in healthy control rats to 6.5±0.5 μmol/g in vehicle-treated CDHFD/NaNO₂ rats, p<0.001). Treatment with either the compound of Formula (I) or the compound of Formula (III) administered as single agents, did not significantly reduce liver hydroxyproline content. Treatment with the combination of the compound of Formula (I) and the compound of Formula (III), reduced liverhydroxyproline by 33% (liver hydroxyproline content was reduced from to 6.5±0.5 μmol/g in vehicle-treated CDHFD/NaNO₂ rats to 4.4±0.5 μmol/g, p<0.01). The effect of combination treatment of the compound of Formula (I) and the compound of Formula (III) to reduce liver hydroxyproline was significantly greater than the compound of Formula (III) administered alone (p<0.05 vs. the compound of Formula (III) alone).

### Quantitative IHC of the Myofibroblast Marker Desmin

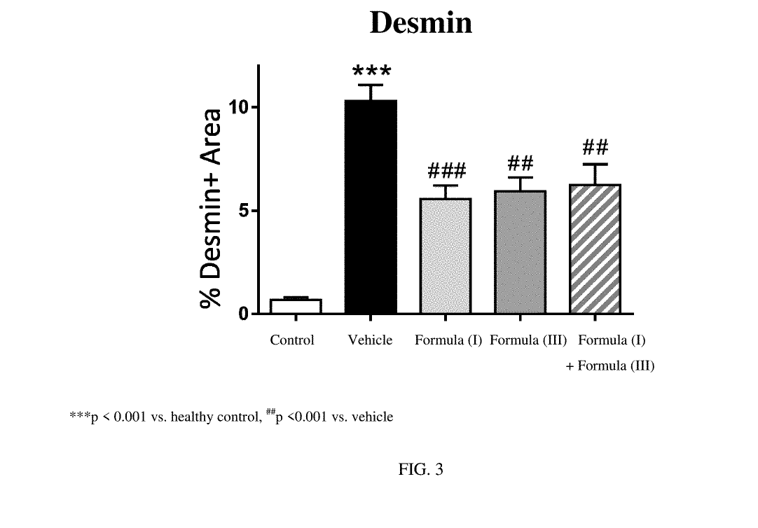

Histological liver sections were stained for desmin, a marker of a marker of activated myofibroblasts. The data are shown in FIG. 3. Desmin staining was quantified by morphometric image analysis and expressed as % desmin marker area. Rats administered CDHFD/NaNO₂ had a 15-fold increase in desmin staining in liver when compared to healthy control rats (liver % desmin marker area increased from 0.7±0.1% in healthy control rats to 10.3±0.8% in vehicle-treated CDHFD/NaNO₂ rats, p<0.001). Treatment with the compound of Formula (I) reduced liver desmin staining by 46% (liver % desmin marker area was reduced from 10.3±0.8% in vehicle-treated CDHFD/NaNO₂ rats to 5.6±0.7%, p<0.001). Treatment with the compound of Formula (III) reduced liver desmin staining by 43% (liver % desmin marker area was reduced from 10.3±0.8% in vehicle-treated CDHFD/NaNO₂ rats to 5.9±0.7%, p<0.001). Treatment with the combination of the compound of Formula (I) and the compound of Formula (III) reduced liver desmin staining by 39% (liver % desmin marker area was reduced from 10.3±0.8% in vehicle-treated CDHFD/NaNO₂ rats to 6.2±1.0%, p<0.001).

### Hepatic Expression of Col1a1 and Timp1

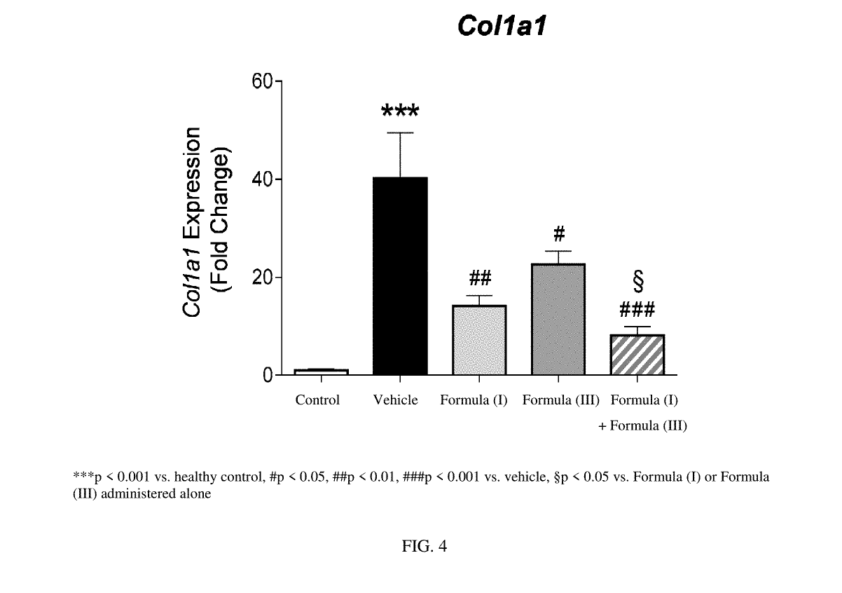

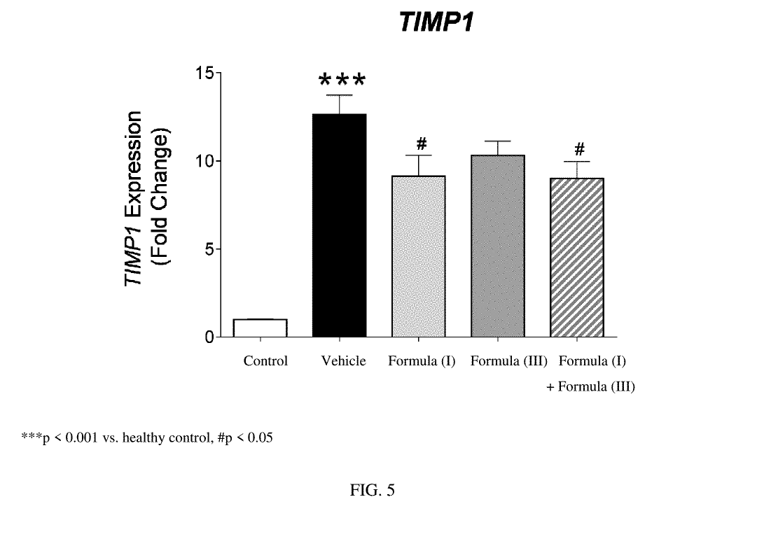

Hepatic expression of the liver fibrosis genes Col1a1 and TIMP-1 were increased by 40-fold fold and 13-fold respectively following CDHFD/NaNO₂ administration when compared to healthy control rats at the end of the 12 week study (p<0.001). The data for Col1a1 are shown in FIG. 4, and the data for TIMP-1 are shown in FIG. 5. Treatment with the compound of Formula (I) reduced Col1a1 induced by CDHFD/NaNO₂ by 65% (p<0.01 vs. vehicle) and reduced and TIMP-1 by 28% (p<0.05 vs. vehicle). Treatment with the compound of Formula (III) reduced hepatic Col1a1 expression induced by CDHFD/NaNO₂ by 44% (p<0.05 vs. vehicle) but did not significantly reduce TIMP-1. Combined treatment of the compound of Formula (I) and the compound of Formula (III) reduced hepatic Col1a1 expression induced by CDHFD/NaNO₂ by 80% (p<0.001 vs. vehicle). The effect of combined treatment of the compound of Formula (I) and the compound of Formula (III) to reduce Col1a1 gene expression was statistically significantly greater compared to either the compound of Formula (I) or the compound of Formula (III) administered alone (p<0.05 vs. vehicle). The effect of combined treatment of the compound of Formula (I) and the compound of Formula (III) to reduce TIMP-1 gene expression was not statistically significantly different compared to the reduction observed by the compound of Formula (I) treatment alone.

In summary, the data from this study demonstrate that the combined treatment with an ASK1 inhibitor and an FXR agonist resulted in greater anti-fibrotic efficacy than either agent administered alone in a rodent model of NASH.

### Example 2. Efficacy in a Mouse Model of NASH

The following study was conducted to evaluate the efficacy of the combination of an ASK1 inhibitor and an FXR agonist in a mouse model of non-alcoholic steatohepatitis (NASH), relative to the efficacy of the individual agents alone in the model. NASH was induced in male C57BL/6 mice by chronic administration of a “fast food” diet (FFD) high in saturated fats, cholesterol and sugars for a total of 10 months, whereas lean control animals were maintained on a normal chow diet. A NASH phenotype was established in FFD mice compared to control mice by 7 months, and was characterized by obesity, hypercholesterolemia, and AST/ALT elevation; and by histological features of NASH such as hepatocellular macrovesicular steatosis and ballooning degeneration. See Charlton M, et al. Fast food diet mouse: novel small animal model of NASH with ballooning, progressive fibrosis, and high physiological fidelity to the human condition. American journal of physiology. Gastrointestinal and liver physiology 2011; 301 (5):G825-34.

After 7 months, FFD mice were subsequently treated with placebo (vehicle), an ASK1 inhibitor (Formula (I)), an FXR agonist (Formula (III)), or with the combination of Formula (I) and Formula (III) for 3 months. Control mice remained on a normal chow diet for the entire 10 month study period. Endpoint analyses included morphometric quantification of liver steatosis (% of steatotic area), liver cholesterol content, serum ALT/AST levels, serum bile acid levels, and nanostring evaluation of gene expression.

### Methods

### Animals

Male, C57BL/6 mice (aged 12 weeks at study inception) and used in the study. All animals were housed under standard vivarium conditions and allowed to acclimate for 7 days prior to study initiation.

### In-Life Experimental Protocol for the FFD Mouse Model

The experimental design is shown in Table 2. Animals were administered a commercially available high fat, high cholesterol diet (D12079B; Research Diets Inc., New Brunswick, N.J.) and drinking water containing 23.1 g fructose (Sigma, F2543) and 17.2 g glucose (Sigma, 49158) per 1000 mL of tap water to represent a fast-food diet (FFD) for a total of 10 months. All study mice were singly caged (1 mouse/cage). Treatment with the compound of Formula (I) or the compound of Formula (III) alone, or the combination of the compounds of Formula (I) and Formula (III) were administered for the final 3 months of the study (month 7-month 10). A separate group of age-matched mice received a standard rodent chow for the entire study duration (Teklad diet TD2014, Indianapolis, Ind.) to represent a normal control group.

The compound of Formula (I) was administered as a 0.15% admixture in the FFD diet (n=x per group), the compound of Formula (III) was administered via oral gavage once a day (10 mg/kg, PO, QD). The vehicle for administration of the compound of Formula (III) was composed of Sodium CMC, 1% w/w Ethanol, 98.5% w/w 50 mM Tris Buffer, pH 8. Vehicle control animals were administered the same vehicle without drug added.

TABLE 2
Experimental Design and Dose Groups

| | | Diet | | Dose |
|---|---|---|---|---|
| Group | n | (months 1-10) | Treatment | (months 7-10) |
| 1 | 7 | Normal Chow | N/A | N/A |
| 2 | 6 | FFD | Vehicle | N/A |
| 3 | 8 | FFD | Formula (I) | 0.15% in chow |
| 4 | 8 | FFD | Formula (III) | 10 mg/kg/day, PO |
| 5 | 9 | FFD | Formula (I) | 0.15% in chow |
| | | | Formula (III) | 10 mg/kg/day, PO |

### Measurement of Hepatic Steatosis

Whole slide-scan images of Hematoxylin & Eosin (H&E) and Picrosirius Red (PSR) stained slides were captured using a Leica SCN400 scanner at 40× magnification. Digital slide images were checked for scanning quality, annotated and exported to appropriate network folders within Leica Digital Image Hub archive. Extent of steatosis was determined on H&E stained tissue sections using the Definiens Developer software package. Analysis parameters allowed for the proper measurement of total tissue cross-sectional area, while excluding optical anomalies and damaged tissue areas. Steatotic lipid vacuoles within the liver parenchyma were observable as areas of low optical density (white). The number and size of these areas were enumerated and the total steatotic area was expressed as a percentage of total liver tissue cross-sectional area. Intrahepatic vessels (such as branches of the portal vein and central vein) were excluded from this analysis based upon vessel size and dimensions. The results of the automated analysis were manually reviewed in order to determine accuracy of the results. Samples failing predetermined QC criteria (inaccurate identification of tissue and inaccurate identification of steatotic area) were excluded from reporting.

### Determination of Total Liver Cholesterol

Tissue samples (25±5 mg, weighed in frozen state) were homogenized and extracted with a water immiscible organic solvent mixture that extracts the free and esterified cholesterol fractions into the organic phase. After centrifugation, an aliquot of the organic upper layer, containing cholesterol and cholesterol esters, was analyzed.

An internal standard solution (cholesterol-d6) and 1 M ethanolic potassium hydroxide solution were added to an aliquot of the appropriate sample dilution. The mixture was incubated at 70° C. for one hour in order to hydrolyze the cholesterol esters to free fatty acids and cholesterol. Afterwards, the reaction mixture was acidified with glacial acetic acid and extracted with hexanes. The hexanes layer was removed, evaporated and reconstituted in acetonitrile. An aliquot of the reconstituted extract was then injected onto a Waters Acquity/AB Sciex QTrap 4000 LC MS/MS system equipped with a C18 reversed phase column. The peak area of the m/z 369 [M-H2O]+→161+ product ion of cholesterol was measured against the peak area of the cholesterol-D6 product ion of m/z 375 [M-H2O]+→167+. Quantitation was performed using a weighted (1/x) linear least squares regression analysis generated from the fortified calibration standards using cholesteryl oleate as reference standard. Calibration standard samples were taken through the same extraction and hydrolysis steps as the tissue samples. Raw data were collected and processed using AB SCIEX software Analyst 1.5.1. Data reduction, weight corrections, correction for cholesteryl oleate to cholesterol hydrolysis and concentration calculations were performed using Microsoft Excel 2013. Final tissue contents are given in mg Total Cholesterol/g Liver Tissue.

### Measurement of Bile Acids

Plasma samples were sent to Metabolon, Inc. (Durham, N.C.) for quantification of primary and secondary bile acids and their conjugates by LC-MS/MS.

### Gene Expression

RNA isolation, reverse transcription and qPCR was run at DC3 Therapeutics, South San Francisco. Livers were homogenized using the Precellys 24 homogenizer according to the manufacturer instructions. RNA was isolated using the E.Z.N.A. HP Total RNA Kit (Omega Biotek #R6812) with DNase I Digestion Set (Omega Biotek #E1091) according to the manufacturer instructions. A Nanostring nCounter XT Reporter CodeSet and Capture ProbeSet were allowed to thaw at room temperature. A master mix was created by adding 70 μL of hybridization buffer to the Reporter CodeSet tube. 8 μL of master mix was then added to each of the 12 hybridization strip tubes. 5 μL of RNA was added to each tube followed by 2 μL of Capture ProbeSet. Tubes were then placed in a pre-heated 65° C. thermal cycler (Veriti, Applied Biosystems) for 16 hours. Hybridization strip tubes were then placed in nCounter Prep Station (NanoString Technologies, Inc. Cat # NCT-PREP-120) with reagents and consumables from the nCounter Master Kit for sample processing and placed in Digital Analyzer (NanoString Technologies, Inc, Cat # NCT-DIGA-120) for data acquisition. Data was analyzed using nSolver analysis software (Nanostring), and presented as fold change

### Results

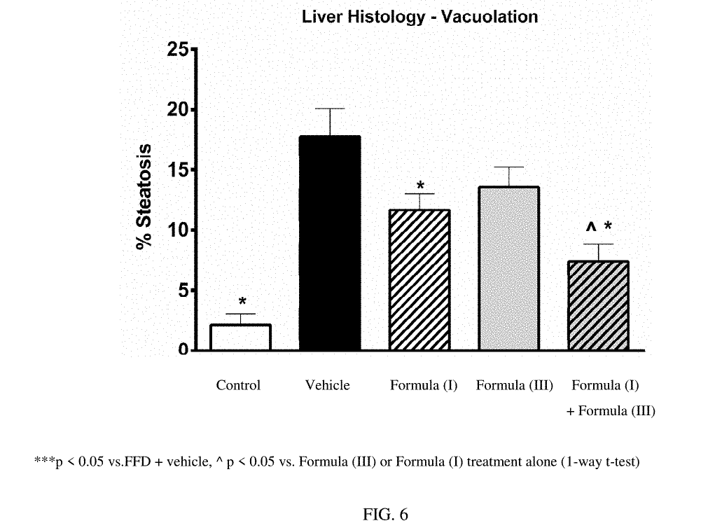

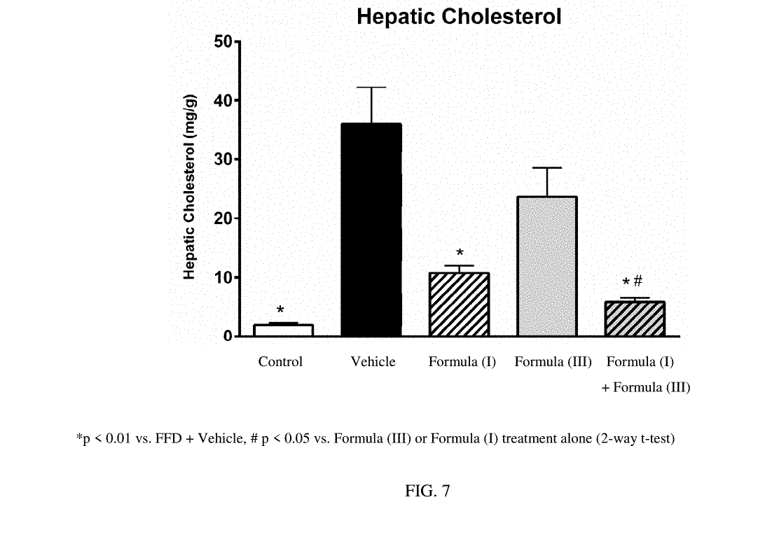

The quantification of H&E stained liver sections demonstrated that the compound of Formula (I), the compound of Formula (III), and the combination of the compounds of Formula (I) and Formula (III) resulted in 39%, 27%, and 75% reduction in steatosis, respectively (FIG. 6). In addition, liver cholesterol content was reduced by 74%, 36% and 88% for the compound of Formula (I), the compound of Formula (III), and the combination of the compounds of Formula (I) and Formula (III), respectively (FIG. 7). Treatment with the combination of the compounds of Formula (I) and Formula (III) resulted in a statistically greater reduction in vacuolation area and in liver cholesterol content compared to treatment with either agent alone.

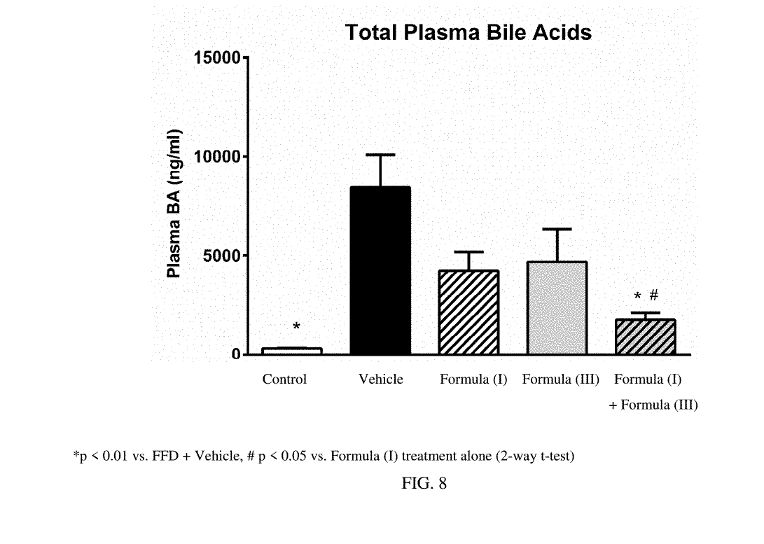

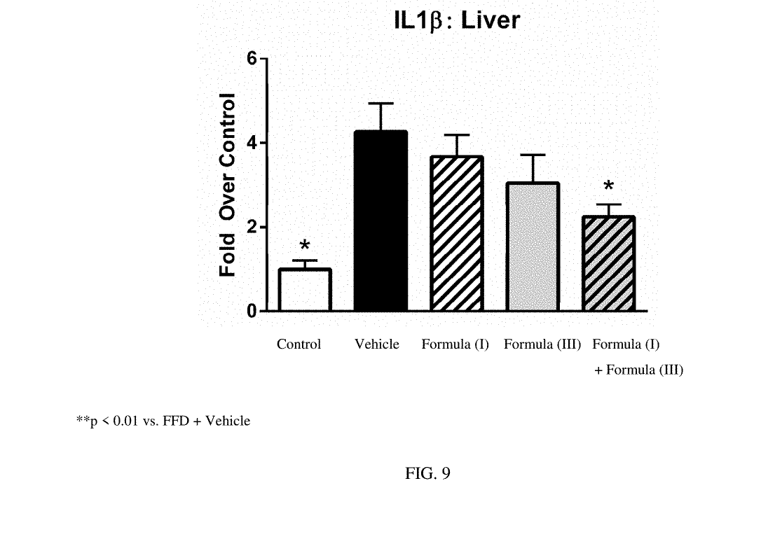

The level of plasma bile acids were significantly elevated in control FFD mice. Administration of the compound of Formula (I), the compound of Formula (III), and the combination of the compounds of Formula (I) and Formula (III) resulted in 52%, 46, and 82% reduction in plasma bile acid levels, respectively (FIG. 8). Treatment with the combination of the compounds of Formula (I) and Formula (III) resulted in the greatest decrease in bile acid levels. In addition, the hepatic expression of the inflammatory gene IL1-β was significantly decreased by treatment with the combination of the compounds of Formula (I) and Formula (III) (FIG. 9).

In summary, the data from this study demonstrate that the combined treatment with an ASK1 inhibitor and an FXR agonist resulted in greater anti-steatotic efficacy than either agent administered alone in a rodent model of NASH.

1. A method of treating and/or preventing a liver disease in a patient in need thereof, comprising administering to the patient a therapeutically effective amount of an ASK1 inhibitor in combination with a therapeutically effective amount of FXR agonist, wherein the ASK1 inhibitor is a compound of Formula (I): 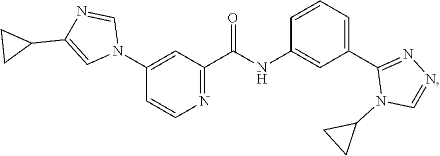 [SMILES: O=C(Nc1cccc(-c2nncn2C2CC2)c1)c1cc(-n2cnc(C3CC3)c2)ccn1]
or a pharmaceutically acceptable salt thereof;
and the FXR agonist is a compound of Formula (III): 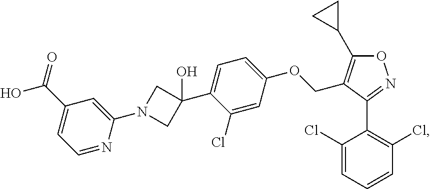 [SMILES: O=C(O)c1ccnc(N2CC(O)(c3ccc(OCc4c(-c5c(Cl)cccc5Cl)noc4C4CC4)cc3Cl)C2)c1]
or a pharmaceutically acceptable salt thereof.

2. A method of treating and/or preventing a liver disease in a patient in need thereof, comprising administering to the patient a therapeutically effective amount of an ASK1 inhibitor in combination with a therapeutically effective amount of FXR agonist, wherein the ASK1 inhibitor is a compound of Formula (I): 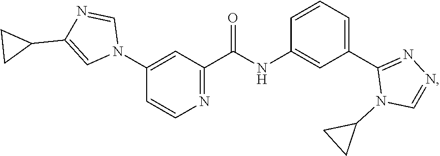 [SMILES: O=C(Nc1cccc(-c2nncn2C2CC2)c1)c1cc(-n2cnc(C3CC3)c2)ccn1]
or a pharmaceutically acceptable salt thereof;
and the FXR agonist is a compound of Formula (IV): 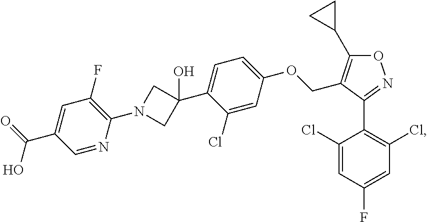 [SMILES: O=C(O)c1cnc(N2CC(O)(c3ccc(OCc4c(-c5c(Cl)cc(F)cc5Cl)noc4C4CC4)cc3Cl)C2)c(F)c1]
or a pharmaceutically acceptable salt thereof.

3. A method of treating and/or preventing a liver disease in a patient in need thereof, comprising administering to the patient a therapeutically effective amount of an ASK1 inhibitor in combination with a therapeutically effective amount of FXR agonist, wherein the ASK1 inhibitor is a compound of Formula (II): 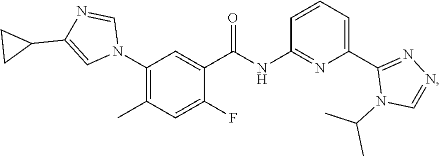 [SMILES: Cc1cc(F)c(C(=O)Nc2cccc(-c3nncn3C(C)C)n2)cc1-n1cnc(C2CC2)c1]
or a pharmaceutically acceptable salt thereof;
and the FXR agonist is a compound of Formula (III): 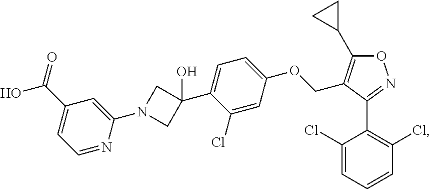 [SMILES: O=C(O)c1ccnc(N2CC(O)(c3ccc(OCc4c(-c5c(Cl)cccc5Cl)noc4C4CC4)cc3Cl)C2)c1]
or a pharmaceutically acceptable salt thereof.

4. A method of treating and/or preventing a liver disease in a patient in need thereof, comprising administering to the patient a therapeutically effective amount of an ASK1 inhibitor in combination with a therapeutically effective amount of FXR agonist, wherein the ASK1 inhibitor is a compound of Formula (II): 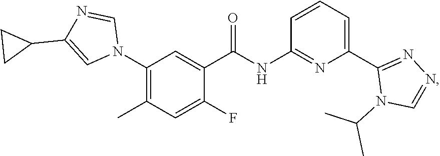 [SMILES: Cc1cc(F)c(C(=O)Nc2cccc(-c3nncn3C(C)C)n2)cc1-n1cnc(C2CC2)c1]
or a pharmaceutically acceptable salt thereof;
and the FXR agonist is a compound of Formula (IV): 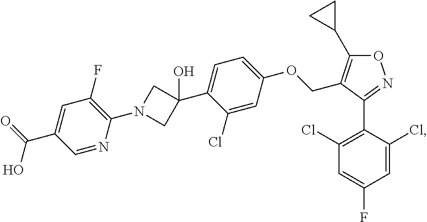 [SMILES: O=C(O)c1cnc(N2CC(O)(c3ccc(OCc4c(-c5c(Cl)cc(F)cc5Cl)noc4C4CC4)cc3Cl)C2)c(F)c1]
or a pharmaceutically acceptable salt thereof.

5. The method of claim 1, wherein the liver disease is non-alcoholic steatohepatitis (NASH).

6. The method of claim 2, wherein the liver disease is non-alcoholic steatohepatitis (NASH).

7. The method of claim 3, wherein the liver disease is non-alcoholic steatohepatitis (NASH).

8. A pharmaceutical composition comprising a therapeutically effective amount of an ASK1 inhibitor and a therapeutically effective amount of a FXR agonist, wherein the ASK1 inhibitor is a compound of Formula (I): 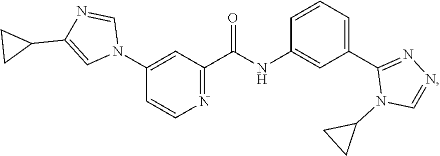 [SMILES: O=C(Nc1cccc(-c2nncn2C2CC2)c1)c1cc(-n2cnc(C3CC3)c2)ccn1]
or a pharmaceutically acceptable salt thereof;
and the FXR agonist is a compound of Formula (III): 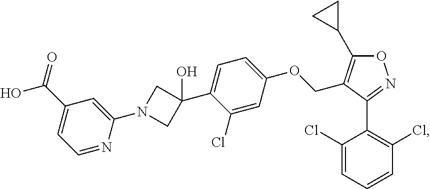 [SMILES: O=C(O)c1ccnc(N2CC(O)(c3ccc(OCc4c(-c5c(Cl)cccc5Cl)noc4C4CC4)cc3Cl)C2)c1]
or a pharmaceutically acceptable salt thereof.

9. A pharmaceutical composition comprising a therapeutically effective amount of an ASK1 inhibitor and a therapeutically effective amount of a FXR agonist, wherein the ASK1 inhibitor is a compound of Formula (I): 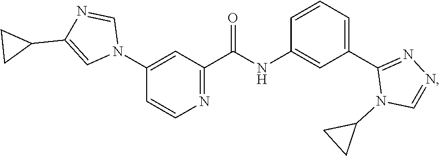 [SMILES: O=C(Nc1cccc(-c2nncn2C2CC2)c1)c1cc(-n2cnc(C3CC3)c2)ccn1]
or a pharmaceutically acceptable salt thereof;
and the FXR agonist is a compound of Formula (IV): 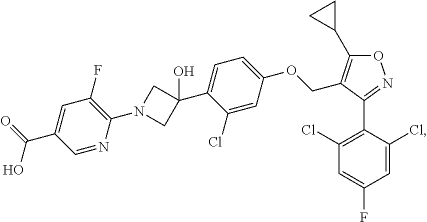 [SMILES: O=C(O)c1cnc(N2CC(O)(c3ccc(OCc4c(-c5c(Cl)cc(F)cc5Cl)noc4C4CC4)cc3Cl)C2)c(F)c1]
or a pharmaceutically acceptable salt thereof.

10. A pharmaceutical composition comprising a therapeutically effective amount of an ASK1 inhibitor and a therapeutically effective amount of a FXR agonist, wherein the ASK1 inhibitor is a compound of Formula (II): 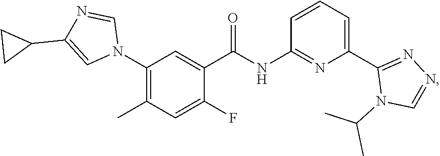 [SMILES: Cc1cc(F)c(C(=O)Nc2cccc(-c3nncn3C(C)C)n2)cc1-n1cnc(C2CC2)c1]
or a pharmaceutically acceptable salt thereof;
and the FXR agonist is a compound of Formula (III): 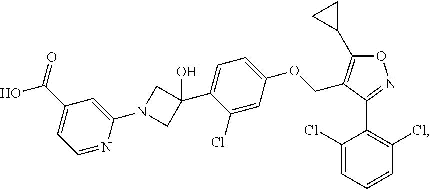 [SMILES: O=C(O)c1ccnc(N2CC(O)(c3ccc(OCc4c(-c5c(Cl)cccc5Cl)noc4C4CC4)cc3Cl)C2)c1]
or a pharmaceutically acceptable salt thereof.

11. A pharmaceutical composition comprising a therapeutically effective amount of an ASK1 inhibitor and a therapeutically effective amount of a FXR agonist, wherein the ASK1 inhibitor is a compound of Formula (II): 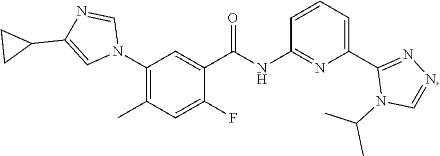 [SMILES: Cc1cc(F)c(C(=O)Nc2cccc(-c3nncn3C(C)C)n2)cc1-n1cnc(C2CC2)c1]
or a pharmaceutically acceptable salt thereof;
and the FXR agonist is a compound of Formula (IV): 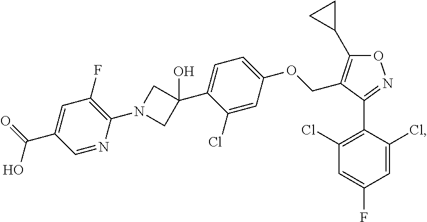 [SMILES: O=C(O)c1cnc(N2CC(O)(c3ccc(OCc4c(-c5c(Cl)cc(F)cc5Cl)noc4C4CC4)cc3Cl)C2)c(F)c1]
or a pharmaceutically acceptable salt thereof.

12. The pharmaceutical composition of claim 8, further comprising a pharmaceutically acceptable carrier.

13. The pharmaceutical composition of claim 9, further comprising a pharmaceutically acceptable carrier.

14. The pharmaceutical composition of claim 10, further comprising a pharmaceutically acceptable carrier.

15. The pharmaceutical composition of claim 11, further comprising a pharmaceutically acceptable carrier.

16. The method of claim 4, wherein the liver disease is non-alcoholic steatohepatitis (NASH).
